(19) United States
(12) Patent Application Publication
(10) Pub. No.: US 2026/0338650 A1
(43) Pub. Date: Oct. 29, 2026

(54) DIRECT AFFINITY TO HYDROPHOBIC INTERACTION CHROMATOGRAPHY METHODS AND SYSTEMS THEREOF

(71) Applicant: Waters Technologies Corporation, Milford, MA (US)

(72) Inventors: Beatrice Muriithi, Attleboro, MA (US); Martin Gilar, Franklin, MA (US); Kevin Wyndham, Worcester, MA (US); Balasubrahmanyam Addepalli, Bellingham, MA (US)

(73) Assignee: Waters Technologies Corporation, Milford, MA (US)

(21) Appl. No.: 19/643,975

(22) Filed: Apr. 10, 2026

Related U.S. Application Data
(60) Provisional application No. 63/794,883, filed on Apr. 25, 2025.

(51) Int. Cl. G01N 1/34; B01D 15/38; G01N 30/02; G01N 30/52; G01N 30/60

(52) U.S. Cl. CPC G01N 1/34; B01D 15/3804; G01N 30/52; G01N 30/6039; G01N 2030/027; G01N 2030/525

(57) ABSTRACT

The present disclosure is directed to methods of performing direct affinity-hydrophobic interaction (HIC) chromatography, wherein there is a direct elution of the sample from the affinity chromatography column to the HIC column. The methods described herein allow for rapid and robust purification of target analytes from heterogeneous samples, and mitigate the need for complicated valve switching, buffer exchange, or other sample manipulation.

### CROSS-REFERENCE TO RELATED APPLICATIONS

This application claims priority from and the benefit of U.S. Provisional Application No. 63/794,883 filed Apr. 25, 2025. The entire contents of this application are incorporated herein by reference.

### FIELD OF INVENTION

The present disclosure relates generally to methods of performing direct affinity chromatography to hydrophobic interaction chromatography, particularly using a direct fluidic connection between the affinity chromatography column and the hydrophobic interaction column.

### BACKGROUND

Chromatography methods are used across the pharmaceutical, biotechnology, and chemical industries for the separation and characterization of target analytes in heterogeneous samples. Multiple chromatography methods may be necessary in a particular workflow. For example, the use of affinity chromatography to isolate a target analyte based on its affinity to an affinity agent, and hydrophobic interaction chromatography to isolate a target analyte based on its affinity to an hydrophobic agent. Typically, the use of both affinity chromatography and hydrophobic interaction chromatography requires separate or complex chromatography systems due to technical incompatibilities, including buffer and pressure incompatibilities. In particular, the different solvent conditions required for affinity chromatography and hydrophobic interaction chromatography greatly increase the processing required for sample purification method requiring both affinity and hydrophobic interaction chromatography.

### SUMMARY OF TECHNOLOGY

Disclosed herein are methods of performing direct affinity-hydrophobic interaction liquid chromatography (affinity-HIC chromatography). The methods allow for the direct elution of a sample from an affinity chromatography column onto an HIC column, without the need for valve switching, buffer exchange, or other sample manipulation. That is, the present technology provides affinity chromatography and HIC columns that are connected in direct fluidic connection, preferably via a zero dead volume union. The technology utilizes an affinity chromatography column having a stationary phase including nonporous polymer particles. Typically, the plurality of nonporous polymer particles have an average particle size between 1.0 μm to 10 μm and a functionalized surface. Said column affords the elution of a target analyte in a small volume, which can then be directly eluted onto the HIC column without the need for buffer exchange or sample concentration. Further, the affinity chromatography columns and the HIC columns used in the disclosed methods have pressure compatibility, eliminating the need for complicated valve switching designs in the liquid chromatography systems. The present invention also includes methods utilizing a gradient mobile phase (i.e., a mobile phase whose composition is adjusted in-experiment between the affinity chromatography step and the HIC chromatography step).

Accordingly, in a first aspect, the present disclosure provides for a method of purifying a target analyte, the method including:
a) loading a sample including the target analyte onto an affinity chromatography column in direct fluidic connection to a hydrophobic interaction chromatography (HIC) column, the affinity chromatography column including:
   a plurality of nonporous polymer particles, wherein each particle within the plurality of nonporous polymer particles comprises a polymer core and a hydrophilic surface on an outer layer of the polymer core; and
   one or more affinity agents conjugated to the hydrophilic surface of each particle of the plurality of nonporous polymer particles; and the HIC column including:
   a plurality of HIC particles including a non-polar surface;
b) eluting the target analyte from the affinity chromatography column with an affinity buffer directly onto the HIC column; and
c) eluting the target analyte from the HIC column with an HIC buffer.
   Typically, the plurality of nonporous polymer particles have an average particle size between 1.0 μm to 10 μm. Typically, the plurality of HIC particles have an average particle size between 1.0 μm to 10 μm.

In some embodiments, the one or more affinity agents are conjugated directly to a surface of each particle of the plurality of nonporous polymer particles. In some embodiments, the one or more affinity agents are conjugated to the surface of each particle of the plurality of nonporous polymer particles covalently.

In some embodiments, the one or more affinity agents are conjugated indirectly to the surface of each particle through one or more conjugating molecules. In some embodiments, the one or more conjugating molecules include streptavidin. In some embodiments, the affinity agent is biotinylated.

In some embodiments, the one or more affinity agents include an immunoglobulin-binding protein, an antibody or antigen-binding fragment thereof, or an oligonucleotide. In some embodiments, the immunoglobulin-binding protein is Protein A, Protein G, Protein A/G, Protein L, or a binding domain thereof. In some embodiments, the antibody or antigen-binding fragment thereof binds to insulin, an AAV capsid, tacrolimus, troponin, IgG, a cytokine, a host cell protein, a dsRNA, or perfluoroalkyl substances (PFAS). In some embodiments, the AAV capsid is AAV1, AAV2, AAV3, AAV4, AAV5, AAV6, AAV7, AAV8, AAV9, AAVrh10, or a synthetic serotype thereof. In some embodiments, the oligonucleotide is a poly-T oligonucleotide. In some embodiments, the plurality of nonporous polymer particles have an average particle size between 1.0 μm to 10 μm.

In some embodiments, the affinity buffer includes sodium phosphate, sodium chloride, ammonium acetate, or ammonium formate. In some embodiments, the affinity buffer has a pH of from about 6.8 to about 8. In some embodiments, the affinity buffer further includes an organic solvent. In some embodiments, the organic solvent is at a concentration of between 1 and 30% by volume. In some embodiments, the organic solvent is at a concentration of between 1 and 20% by volume. In some embodiments, the organic solvent is at a concentration of between 1-10% by volume. In some embodiments, the concentration of organic solvent is insufficient to denature the affinity agent. In some embodiments, the organic solvent is ethanol or acetonitrile.

In some embodiments, the affinity chromatography column and the HIC column are connected to a high-performance liquid chromatography (HPLC) system, ultra-high performance liquid chromatography (UHPLC) system, or fast protein liquid chromatography (FPLC) system.

In some embodiments, the method further includes the step of detecting the target analyte with a detector after eluting the target analyte from the HIC column. In some embodiments, the detector is an ultraviolet spectroscopy detector, a fluorescence spectroscopy detector, a multi-angle light scattering detector, a charged aerosol detector, and/or a mass spectrometry detector. In some embodiments, the direct fluidic connection is a zero dead volume union.

In a second aspect, the present disclosure further provides for a chromatographic system including:
(a) an affinity chromatography column and an hydrophobic interaction chromatography (HIC) column, wherein the affinity chromatography column and the HIC column are connected via a zero dead volume union, wherein the HIC column is positioned downstream of the affinity chromatography column; wherein the affinity chromatography column includes:
   a plurality of nonporous polymer particles, wherein each particle within the plurality of nonporous polymer particles comprises a polymer core and a hydrophilic surface on an outer layer of the polymer core; and
   one or more affinity agents conjugated to the hydrophilic surface of the nonporous polymer particle; and the HIC column includes:
   a plurality of HIC particles having a non-polar surface;
(b) a column injector positioned upstream of the affinity chromatography column; and
(c) tubing in fluidic connection with the HIC column positioned downstream of the HIC column.
   Typically, the plurality of nonporous polymer particles have an average particle size between 1.0 μm to 10 μm. Typically, the plurality of HIC particles have an average particle size between 1.0 μm to 10 μm.

In some embodiments, the one or more affinity agents are conjugated directly to a surface of each particle of the plurality of nonporous polymer particles. In some embodiments, the affinity agent is conjugated to the surface of each particle of the plurality of nonporous polymer particles covalently.

In some embodiments, the affinity agent is conjugated indirectly to the surface of each particle through one or more conjugating molecules. In some embodiments, the one or more conjugating molecules include streptavidin. In some embodiments, the affinity agent is biotinylated.

In some embodiments, the one or more affinity agents include an immunoglobulin-binding protein, an antibody or antigen-binding fragment thereof, or an oligonucleotide. In some embodiments, the immunoglobulin-binding protein is Protein A, Protein G, Protein A/G, Protein L, or a binding domain thereof. In some embodiments, the antibody or antigen-binding fragment thereof binds to insulin, an AAV capsid, tacrolimus, troponin, IgG, a cytokine, a host cell protein, a dsRNA, or perfluoroalkyl substances (PFAS). In some embodiments, the AAV capsid is AAV1, AAV2, AAV3, AAV4, AAV5, AAV6, AAV7, AAV8, AAV9, AAVrh10, or a synthetic serotype thereof. In some embodiments, the oligonucleotide is a poly-T oligonucleotide.

In some embodiments, the chromatographic system further includes a detector in fluid communication with the tubing downstream of the HIC chromatography column.

### DEFINITIONS

In order that the methods and technology may be more readily understood, certain terms are first defined. In addition, it should be noted that whenever a value or range of values of a parameter are recited, it is intended that values and ranges intermediate to the recited values are also part of this disclosure. The word “about”, if not defined otherwise, means ±5%. It is also to be noted that as used herein and in the claims, the singular forms “a” and “the” include plural references unless the context clearly dictates otherwise.

As used herein, the term “direct fluidic connection” refers to a flow path between one or more column bodies, column injectors, detectors, and/or tubing connecting said elements, that is uninterrupted by a switching valve.

As used herein, the term “zero dead volume union” refers to a union between one or more elements having a direct fluidic connection, wherein the union introduces 1 μL or less of volume to the flow path. A zero dead volume union can be introduced between any two elements having a direct fluidic connection. In preferred embodiments of the present technology, a zero dead volume union is introduced between an affinity chromatography column and an hydrophobic interaction chromatography (HIC) column having a direct fluidic connection.

As used herein, the term “low volume union” refers to a union between one or more elements having a direct fluidic connection, wherein the union introduces 30 μL or less of volume to the flow path, preferably 20 μL or less, more preferably 10 μL or less. A low volume union can be introduced between any two elements having a direct fluidic connection. In some embodiments of the present technology, a low volume union is introduced between an affinity chromatography column and an HIC column having a direct fluidic connection.

As used herein, the term “direct affinity-hydrophobic interaction chromatography” or “direct affinity-HIC”, used interchangeably, refers to a method of performing chromatography wherein a sample is first separated via an affinity chromatography column and then directly transferred onto an HIC column, and separated by the HIC column. In the direct affinity-HIC methods described herein, the affinity chromatography column is in direct fluidic connection to the hydrophobic interaction chromatography column. That is, any sample injected onto the affinity chromatography column flows through both the affinity chromatography column and the hydrophobic interaction chromatography column, as shown in FIG. 1A.

As used herein, the term “eluting the target analyte from the affinity chromatography column” refers to the use of an elution buffer and elution method (such as, e.g., a gradient elution, a single injection elution, or a step elution) to elute analytes bound to the affinity column. In some embodiments, the analytes are eluted in a single narrow peak having a peak volume of between 5-20 μL.

As used herein, the term “conjugate” refers to the linkage of two molecules formed by the chemical bonding of a reactive functional group of one molecule with an appropriately reactive functional group of another molecule. Nonporous polymer particles may have one or more affinity reagents conjugated to the surface of said particles. For example, one or more affinity agents, such as Protein A, may be conjugated directly to a particle via an interaction with an epoxide on the surface of the particle. Alternatively, an affinity agent may be indirectly conjugated to the surface of the nonporous polymer particles via a linker (such as a polyethylene glycol (PEG) linker) or via an interaction with a streptavidin molecule. For the latter instance, one or more streptavidin molecules are conjugated directly to a particle via an interaction with an epoxide on the surface of the particle, which can then bind, via an ionic interaction, to a biotinylated affinity agent.

As used herein, the term “antibody” refers to an immunoglobin molecule that specifically binds to, or is immunologically reactive with, a particular antigen. This includes polyclonal, monoclonal, genetically engineering, and otherwise modified forms of antibodies, including but not limited to chimeric antibodies, camelids, monobodies, humanized antibodies, heteroconjugate antibodies (e.g., bi-, tri-, and quad-specific antibodies, diabodies), and antigen-binding fragments of antibodies, including, for example, Fab′, F(ab′)2, Fab, Fv, and scFv fragments. Unless otherwise indicated, the term “monoclonal antibody” is meant to include both intact molecules as well as antibody fragments that are capable of specifically binding to a target protein. As used herein, the Fab and F(ab′)2 fragments refer to antibody fragments that lack the Fc portion of an intact antibody.

As used herein, the term “polyclonal antibody” refers to an antibody or a population of antibodies that has specificity to one or more antigens (such as, e.g., host cell proteins from a host cell line). A population of polyclonal antibodies recognize one or more distinct epitopes of the one or more antigens.

As used herein, the term “antigen-binding fragment” refers to one or more fragments of an antibody that retain the ability to specifically bind to a target antigen. The antigen-binding function of an antibody can be performed by fragments of a full-length antibody. The antibody fragments can be, for example, a Fab, F(ab′)₂, scFv, a camelid, an affibody, a nanobody, an aptamer, or a domain antibody.

As used herein, the term “bispecific antibody” refers to an antibody that is capable of binding at least two different antigens.

The term “nonporous” or “nonporous core” as used herein, refers to a material or a material region (e.g., the core) that has a pore volume that is less than 0.1 cc/g. Preferably, nonporous polymer cores have a pore volume that is less than 0.10 cc/g (e.g., 0.05 cc/g), and preferably less than 0.02 cc/g, in some embodiments. Pore volume is determined using methods known in the art based on multipoint nitrogen sorption experiments (Micromeritics ASAP 2400; Micromeritics Instruments Inc., Norcross, GA).

### BRIEF DESCRIPTION OF THE DRAWINGS

FIGS. 1A-1C depicts a liquid chromatography system and flow path for performing direct affinity-HIC according to embodiments of the technology.

FIG. 1A shows the flow path across an LC system set up according to embodiments of the technology.

FIG. 1B provides an enlarged view of the zero dead volume or low volume union between an affinity chromatography column and an HIC column.

FIG. 1C provides an exemplary zero dead volume union.

### DETAILED DESCRIPTION

Disclosed herein are methods of performing tandem affinity chromatography and hydrophobic interaction chromatography (HIC) using a direct elution method from the affinity chromatography column to the HIC column (direct affinity-hydrophobic interaction chromatography; or alternatively direct affinity-HIC).

In conventional technologies, the use of different modes of separation (i.e., affinity chromatography and HIC chromatography) on a single sample requires the use of either valve switching or sample isolation from the first mode of separation (also known as a dimension). The former requires liquid chromatography systems with valve and trap configurations that can be used to first capture (‘trap’) an analyte of interest separated by a first dimension and divert its flow path to the second mode of separation. In these configurations, the modes of separation (dimensions) are decoupled-that is only the trapped analytes from the first dimension are transferred to and separated by the second dimension. Thus, the ability to separate multiple target analytes is limited, in part, by the system configuration and the efficiency of the valve/trap apparatuses. An alternative to valving is to first collect a sample fraction from a first mode of separation, and subsequently injecting the sample fraction onto the second mode of separation. This approach requires manual manipulation of small sample volumes, which can complicate workflows and introduce user error. These approaches are necessary as direct connection of one column type to another in the absence of a valve would result in either many peaks or single broad peaks being eluted from the first column into the second column, thereby reducing separation performance of the second column.

The direct affinity-HIC methods described herein circumvent the aforementioned issues with two-dimensional chromatography while permitting separation of a sample using two chromatographic modes of separation.

In particular, the affinity chromatography columns described herein result in a single load peak from the load step (i.e., a single peak of analytes, wherein the single peak of analytes is composed of one or more analytes that do not bind to the affinity column) and a single, elution peak from the elution step (i.e., a single peak of analytes, wherein the single peak of analytes is composed of one or more analytes), which is on the order of a standard HPLC/UHPLC injection (i.e., between 5-20 μL). Further, the affinity chromatography columns used in the instant technology have pressure compatibility with the HIC columns used herein. Thus, the peaks (the one or more analytes which elute together in either the load step or the elution step) that elute from the affinity chromatography column can be eluted directly onto the HIC column without the need for valving or fraction collection. In doing so, both the analytes from the loading step and the elution step are subsequently separated by the HIC column. The affinity chromatography step of a method described herein includes an affinity buffer solution, and the HIC chromatography step of a method described herein includes an HIC buffer.

### Affinity Chromatography Materials and Columns Thereof

The present technology utilizes affinity chromatography columns for the purification and isolation of a target analyte. The affinity chromatography columns are suitable for use in a high-performance liquid chromatography (HPLC) system or an ultra-high performance liquid chromatography (UHPLC) system and are designed for robust on-column affinity capture at the high pressures and flow conditions of said systems.

### Affinity Chromatography

A sample to be separated by a method disclosed herein may first be separated by an affinity chromatography column. Affinity chromatography serves to separate one or more components of a sample based on the strength of interaction between the component and an affinity agent included with the affinity chromatography column. The strength of these interactions can be controlled by, e.g., the pH of the mobile phase (i.e., the pH of the buffer). Affinity chromatography columns of the present disclosure are suitable for use in a high-performance liquid chromatography (HPLC) system or an ultra-high performance liquid chromatography (UHPLC) system and are designed for robust on-column affinity capture at the high pressures and flow conditions of said systems.

### Affinity Chromatography Materials

The affinity chromatography columns used in the methods disclosed herein include nonporous particles, which provide high surface area for conjugation of affinity agents and can withstand the pressures of HPLC and UHPLC systems. As such, in one aspect the affinity chromatography columns include a plurality of nonporous particles having an average particle size between 1.0 μm to 10 μm. In a preferred embodiment, the nonporous particles are nonporous polymer particles. In some embodiments, each particle within the plurality of particles is highly spherical with a smooth surface. In some embodiments, each particle within the plurality of particles is highly spherical with a bumpy, convex surface. Such materials have surface areas (measured in m²/g) that are close to their theoretical values. The theoretical surface area for a nonporous smooth sphere is equal to 6/{particle diameter x particle density}. For example, 1 μm polymer particles with a density of approximately 1 g/mL has a theoretical surface area of 6 m²/g, a 3.5 μm polymer particle with the same density has a theoretical surface area of 1.7 m²/g, and a 7 μm polymer particle with same density has a theoretical surface area of 0.9 m²/g.

The particles for use in the methods described herein are nonporous. While some pores or porosity may be incorporated within the particles as discontinuities or as microporosity, nonporous particles are those particles having a pore volume that is less than 0.1 cc/g of the material forming the particle. Preferably, nonporous particles have a pore volume that is less than 0.10 cc/g (e.g., 0.05 cc/g), and preferably less than 0.02 cc/g, in some embodiments. Pore volume is determined using methods known in the art based on multipoint nitrogen sorption experiments (Micromeritics ASAP 2400; Micromeritics Instruments Inc., Norcross, GA). Without wishing to be bound by theory, it is believed that the use of nonporous particles is advantageous as it removes diffusion of analytes into pores of the particles, thereby by improving kinetics of the binding and eluting steps of affinity chromatography.

The nonporous particles described herein have an average particle size of between 1 to 10 μm. In some embodiments, the particle size is about 1.7 μm. In some embodiments, the particle size is about 3.5 μm. In some embodiments, the particle size is about 7 μm.

The size (e.g., less than 10 microns), shape (e.g., spherical), and surface area (e.g., nonporous smooth or nonporous bumpy convex) create a form factor useful for affinity chromatography and affinity chromatography columns used in conjunction with HPLC and UHPLC systems. These systems operate under high pressures (e.g., typically greater than 3,000 psi, such as, for example, 5,000 psi, 10,000 psi, 12,000 psi, 15,000 psi and so forth). Therefore, the particles used herein are rigid particles such that the form factor is retained under HPLC and UHPLC operating conditions.

As used herein, the term “rigid particle,” as used herein, refers to the strength of the particle to withstand applied pressures under flow conditions. A rigid particle appears visually undamaged (i.e., maintains the same form factor without breaking, crushing, or alteration) in a scanning electron microscope image after exposure to pressures of 3,500 psi, wherein less than 10% of the observed particles are visually damaged. In addition, particles in a packed bed that are broken or deformed result in reduced flow and increased pressure as one would predict using the Kozeny-Carmen equation. Broken or deformed particles in a packed bed can increase pressure beyond levels suitable for use in HPLC or UHPLC.

Materials that meet the form factor requirements for forming a core (e.g., center or base) of the particles used herein include polymers, in particular organic polymers. Thus, in some embodiments, the nonporous particles include a nonporous polymer core. In some embodiments, the nonporous polymer core is divinylbenzene (DVB), for example divinylbenzene 80%. In some embodiments, the nonporous polymer core is formed to include two or more polymers. For example, in some embodiments the nonporous polymer cores include both DVB and polystyrene. In certain embodiments, the nonporous polymer cores can be manufactured to include a gradient in the polymer composition. For example, the inner portion of the core can be formed of 100% of first polymer (i.e., polymer A) and an outer portion of the core can be formed of 100% or some percentage greater than 0% of a second polymer (i.e., polymer B). Radially from the inner portion to the outer portion of the core, the percentage of polymer A and polymer B can vary to form the gradient in polymer composition. Other embodiments of nonporous polymer cores and particles suitable for use with the present technology are described in U.S. Patent Publication No. 2019/0322783, incorporated herein by reference.

Other nonporous materials can be utilized as long as the form factor of the particles can be maintained under the operating conditions of HPLC or UHPLC. That is, other materials, such as silica, metal oxides, hybrid inorganic-organic materials, or combinations thereof may be used to create nonporous spherical particles having an average particle size of less than 10 microns and the rigidity to retain form factor under high operating pressures (e.g., greater than 3000 psi).

### Affinity Agents

To form particles useful for affinity chromatography, the outer surface of the affinity particle is conjugated, either directly or indirectly, to an affinity agent.

In embodiments for direct conjugation, the outer surface of the nonporous polymer core includes a hydrophilic surface, such as, for example, an epoxide. One or more affinity agents can be directly conjugated to the hydrophilic surface. The hydrophilic surface (or hydrophilic layer, used interchangeably herein) can be formed of a polymer, molecule or siloxane that has a high density of hydrophilic groups (e.g., hydroxyls, PEG, sugars or carbohydrates). The immobilization of these hydrophilic groups can occur by condensation (ester, amid, silanol, sily ether), polymerization (methacrylates, acrylates, styryl) epoxy activation (epihydrochlorin), or ether formation (direct attachment of PEG or carbohydrate groups by ether formation). Further examples include (3-glycidyloxypropyl)trimethoxysilane, (3-glycidyloxypropyl)triethoxysilane, polyacrylate, poly(methyl acrylate), and combinations thereof. Additionally or alternatively, these may include glycidol, glyceroltriglycidyl ether, and combinations thereof.

An affinity agent can be directly conjugated to the hydrophilic surface of the nonporous particle via linkers and methods known in the art, and described in Hermanson G, “Bioconjugate Techniques” 3^{rd} Edition, July 2013).

In embodiments for indirect conjugation, one or more streptavidin molecules are first directly conjugated to the hydrophilic surface of the nonporous particles as described above. Due to the strong affinity between biotin and streptavidin, the streptavidin molecules provide a binding site for biotinylated affinity agents, providing a functionalized particle with a specific affinity (based on the affinity of the affinity agent).

Various affinity agents are suitable for use in the disclosed methods. These include immunoglobulin-binding proteins, antibodies or antigen-binding fragments thereof, oligonucleotides and nucleic acids, or other ligand-binding proteins or peptides. In embodiments wherein the affinity agent is indirectly conjugated to the particle, the affinity agent must be biotinylated.

### Immunoglobulin-Binding Affinity Agents

In one aspect, the affinity agent is an immunoglobulin-binding protein. The immunoglobulin-binding protein provides accessible binding sites for an immunoglobulin, i.e., an antibody, provided that said antibody includes a conserved region that binds to the immunoglobulin-binding protein. In one embodiment, the immunoglobulin-binding protein is Protein A. In another embodiment, the immunoglobulin-binding protein is Protein G. In other embodiments, the immunoglobulin-binding protein is Protein A/G or Protein L. In some embodiments, the immunoglobulin-binding protein is directly conjugated to the surface of the nonporous particle. In some embodiments, the immunoglobulin-binding protein is indirectly conjugated to the surface of the nonporous particle. In embodiments wherein the immunoglobulin-binding protein is indirectly conjugated to the surface of the nonporous particle, the immunoglobulin-binding protein is a biotinylated immunoglobulin-binding protein. For example, a biotinylated Protein A, a biotinylated Protein G, a biotinylated Protein A/G, or a biotinylated Protein L.

Most immunoglobulins (Ig) consist of four polypeptide chains: two identical heavy chains and two identical light chains that are connected by disulfide bonds. Within a given heavy chain or light chain, there is both a variable and a constant region. The constant region, which includes 2-4 constant domains (depending on isotype), is highly conserved within a given isotype. As such, immunoglobulin-binding proteins that bind to a portion of the constant region are suitable for affinity capture of antibodies independent of the antibody's target antigen.

Immunoglobulin-binding proteins suitable for use in the present technology may exhibit strong binding affinity to the Fc portion of an antibody. This binding affinity can vary in strength by both isotype and species. For example, Protein A exhibits strong binding affinity to IgG isotypes but variable to no binding affinity to IgA, IgD, IgE, and IgM isotypes. Even within the IgG isotype, different subclasses can exhibit varied binding affinity. Protein A has high binding affinity to human IgG1, IgG2, and IgG4, but very weak binding affinity to IgG3. By contrast, Protein A binds to murine IgG3 but not to IgG1. Other examples of immunoglobulin-binding proteins, such as Protein G, have high binding affinity to all four subclasses of IgG. Methods for characterizing protein-protein interactions, including binding affinities across a range of environmental conditions, are well known in the art.

### Antibody Affinity Agents

In one aspect, the affinity agent is an antibody or antigen-binding fragment thereof. In some embodiments, the antibody or antigen-binding fragment thereof is a polyclonal antibody, a monoclonal antibody, a single-chain variable fragment (scFv), a nanobody, a monobody, a single domain antibody, a bispecific antibody, or a camelid. In some embodiments, the antibody or antigen-binding fragment thereof is an IgG, IgM, IgA, IgE, or IgD isotype. The antibody or antigen-binding fragment thereof may be derived from a human, mouse, rabbit, goat, or other species. In some embodiments, the antibody is a humanized antibody. In yet other embodiments, the antibody or antigen-binding fragment thereof is a biotinylated antibody or antigen-binding fragment thereof. That is, the biotinylated antibody or antigen-binding fragment thereof is a biotinylated polyclonal antibody, a biotinylated monoclonal antibody, a biotinylated scFv, a biotinylated nanobody, a biotinylated monobody, a biotinylated single domain antibody, a biotinylated bispecific antibody, or a biotinylated camelid.

In some embodiments, the affinity agent is an antibody or antigen-binding fragment thereof that specifically binds to insulin. In some embodiments, the antibody or antigen-binding fragment thereof that specifically binds to insulin is a biotinylated antibody or antigen-binding fragment thereof. In some embodiments, the affinity agent is an antibody or antigen-binding fragment thereof that specifically binds to AAV9. In some embodiments, the antibody or antigen-binding fragment thereof that specifically binds to AAV9 is a biotinylated antibody or antigen-binding fragment thereof. In some embodiments, the affinity agent is an antibody or antigen-binding fragment thereof that specifically binds to AAV2. In some embodiments, the antibody or antigen-binding fragment thereof that specifically binds to AAV2 is a biotinylated antibody or antigen-binding fragment thereof. In some embodiments, the affinity agent is an antibody or antigen-binding fragment thereof that specifically binds to an AAV capsid, such as AAV1, AAV2, AAV3, AAV4, AAV5, AAV6, AAV7, AAV8, and/or AAV9. In some embodiments, the antibody or antigen-binding fragment thereof that specifically binds to an AAV capsid is a biotinylated antibody or antigen-binding fragment thereof.

In some embodiments, the affinity agent is an antibody or antigen-binding fragment thereof that specifically binds to tacrolimus. In some embodiments, the antibody or antigen-binding fragment thereof that specifically binds to tacrolimus is a biotinylated antibody or antigen-binding fragment thereof. In some embodiments, the affinity agent is an antibody or antigen-binding fragment thereof that specifically binds to troponin. In some embodiments, the antibody or antigen-binding fragment thereof that specifically binds to troponin is a biotinylated antibody or antigen-binding fragment thereof. In some embodiments, the affinity agent is an antibody or antigen-binding fragment thereof that specifically binds to IgG. In some embodiments, the antibody or antigen-binding fragment thereof that specifically binds to IgG is a biotinylated antibody or antigen-binding fragment thereof. In some embodiments, the affinity agent is an antibody or antigen-binding fragment thereof that specifically binds to a cytokine. In some embodiments, the antibody or antigen-binding fragment thereof that specifically binds to a cytokine is a biotinylated antibody or antigen-binding fragment thereof. In some embodiments, the affinity agent is an antibody or antigen-binding fragment thereof that specifically binds to perfluoroalkyl substances (PFAS). In some embodiments, the antibody or antigen-binding fragment thereof that specifically binds to PFAS is a biotinylated antibody or antigen-binding fragment thereof. In some embodiments, the affinity agent is an antibody or antigen-binding fragment thereof that specifically binds to a host cell protein (HCP). In some embodiments, the antibody or antigen-binding fragment thereof that specifically binds to a HCP is a biotinylated antibody or antigen-binding fragment thereof.

As used herein, the term “host cell protein” refers to process-related proteinaceous impurities present in a host cell culture or host cell line used during biopharmaceutical manufacturing and production.

### Oligonucleotide and Nucleic Acid Affinity Agents

In another aspect, the affinity agent is an oligonucleotide, nucleic acid, or oligomer. In some embodiments, the oligonucleotide can range from 5-50 nucleotides. In some embodiments, the nucleotide includes 25 nucleotides. Any or all of the nucleotides in a particular oligonucleotide or nucleic acid species can further be modified using methods known in the art, including biotinylating of the oligonucleotide or nucleic acid species. In particular, an oligonucleotide can be biotinylated on the 5′ or 3′ end.

Oligonucleotides suitable as affinity agents may be presented by the following Formula I:

B₀₋₁(Xₙ)ₚB′₀₋₁ (Formula I);
wherein B and B′ are independently a biotin group,
X is independently a nucleotide, nucleoside, or derivative thereof, including but not limited to adenosine, thymidine, guanosine, and cytidine;
n is independently 1-50; and
P is independently 1-50.

In some embodiments of Formula I, at least one of B or B′ is present (i.e., B or B′ is 1). In some embodiments, B is 1 and B′ is 0. In some embodiments, B is 0 and B′ is 1. In some embodiments, both B and B′ are 1. In some embodiments, both B and B′ are 0.

In some embodiments of Formula I, B is 1, X is thymidine, n is 25, p is 1, and B′ is 0. In some embodiments of Formula I, B is 0, X is thymidine, n is 25, p is 1, and B′ is 1. The resultant 5′-biotinylated or 3′-biotinylated oligonucleotide includes 25 thymidine units (i.e., a 25-mer of thymidine or dT₂₅).

In some embodiments, the affinity agent is a biotinylated oligonucleotide of Formula I. In some embodiments, the biotinylated oligonucleotide sequence is complementary to a target analyte sequence.

In one aspect, any nucleic acid-based affinity agent can be used, including biotinylated nucleic acid affinity agents and oligonucleotide affinity agents. The oligonucleotide may include deoxyribonucleic acids (DNA), ribonucleic acids (RNA), or a combination thereof. DNA oligonucleotides include the nucleotides cytidine, guanosine, adenosine, and thymidine. RNA oligonucleotides include the nucleotides cytidine, guanosine, adenosine, and uridine. In some embodiments, the oligonucleotide may include nucleic acid analogues (i.e., non-naturally occurring nucleic acids or analogues thereof). Examples of nucleic acid analogues include peptide nucleic acids, locked nucleic acids, glycol nucleic acids, threose nucleic acids, hexitol nucleic acids. Nucleic acid analogues are further reviewed in Wang et al., Molecules (2023) 28(20): 7043.Oligonucleotides may further be modified at the nucleobase, sugar, or phosphodiester backbone with an array of chemical modifications which are further reviewed in Epple et al., Emerg. Top. Life. Sci. (2021) 5(5): 691-697. In yet other embodiments, the nucleic acid affinity agent is an aptamer having specificity to a target analyte, including a biotinylated aptamer. Methods of biotinylating oligonucleotides, including modified oligonucleotides or oligonucleotides including non-naturally occurring nucleic acid analogues, are well known in the art and would be readily understood by a person of ordinary skill.

### Affinity Chromatography Column Body

The affinity chromatography materials, i.e., the nonporous particles conjugated to an affinity agent, are typically packed into a chromatographic column, thereby resulting in an affinity chromatography column. The column body is typically formed of a metal or a metal alloy, e.g., titanium or stainless steel.

In some embodiments, an alkylsilyl coating or other high-performance surface (HPS) is provided to limit or reduce non-specific binding of a sample with the walls or interior surfaces of the column body. Without wishing to be bound by any particular theory, it is believed than an alkylsilyl coating covering metal surfaces prevents or otherwise minimizes contact between fluids passing through the column. The alkylsilyl coating can be applied to the interior surfaces defining what is known as a wetted path of the column. A metal wetted path includes all surfaces formed from metal that are exposed to fluids during operation of the chromatographic column. The metal wetted path includes not only the column body walls but also metal frits disposed within the column. The coating may be applied not only to the wall of the column body but also to the frits.

In general, the alkylsilyl coating is applied through a vapor deposition technique. Vaporized precursors are charged into a reactor in which the part to be coated is located. These vaporized precursors react on the surfaces of the part to be coated to form a first layer of deposited material. The vapor deposition can be applied in a stepwise function to apply a number of layers of deposited material to the surfaces to grow a thickness of the coating and/or to apply layers of different materials (e.g., alternating between a first and second material) to form the coating.

In some embodiments, the alkylsilyl coating is applied to other portions of the liquid chromatography system. For example, the alkylsilyl coating can be applied to metal components residing upstream and downstream of the column. Specifically, the alkylsilyl coating can be applied to an injector of the liquid chromatography system and to post column tubing and connectors (e.g., tubing and connectors leading from the column to downstream components such as detectors). Further, the affinity chromatographic columns of the present technology do not require the addition of additional organic modifiers to reduce non-specific binding. Typically, the addition of an organic modifier (e.g., acetonitrile) may be necessary with sorbents used in affinity chromatography to reduce non-specific binding. Due to the already low non-specific binding of the columns of the present technology, no organic modifier is necessary.

In one embodiment, the alkylsilyl coating includes a hydrophilic, non-ionic layer of polyethylene glycol silane. In another embodiment, the alkylsilyl coating is formed from one or more of the following precursor materials bis(trichlorosilyl)ethane or bis(trimethoxysilyl)ethane. Other embodiments of alkylsilyl coatings suitable for use with the present technology are described in U.S. Patent Publication No. 2019/0086371 and U.S. Application Publication No. 2022/0118443.

### Affinity Buffers

A number of buffers are suitable for use with the methods provided herein. Affinity buffers suitable for use in liquid chromatography are known in the art and could be determined by a person of ordinary skill in the art. In some embodiments, the wash buffer has a pH of between 6.0 to 8.0. In some embodiments, the wash buffer has a pH of between 6.0 to 6.5, 6.5 to 7.0, 7.0 to 7.5, or 7.5 to 8.0. In some embodiments, the affinity buffer includes sodium phosphate, ammonium acetate, or ammonium formate. For example, but not by way of limitation, a suitable wash buffer may be 50-100 mM sodium phosphate, pH 7.2 to 7.4 or 100-200 mM ammonium formate pH 6.5. Additional washing buffers and concentrations thereof are described in the examples below.

In some embodiments, the wash buffer further includes an organic solvent. Examples of organic solvents suitable for use include acetonitrile and ethanol. In some embodiments, the concentration of the organic solvent is between 0-20% by volume or any number between said range (e.g., between 1-10%).

### Hydrophobic Interaction Chromatography (HIC)

Hydrophobic interaction chromatography (also referred to as HIC herein) is a variant of normal phase liquid chromatography which separates one or more analytes based on their interaction with a charged stationary phase. HIC specifically utilizes a non-polar, hydrophobic stationary phase, and a polar (e.g., high aqueous content or high ionic strength) mobile phase. Methods of the present disclosure include performing HIC on the eluent of an affinity chromatography column with a HIC chromatography column. HIC may be performed using a HIC buffer solution. In some embodiments, HIC may be used to purify a target analyte from a sample. In some embodiments, the target analyte includes an antibody drug conjugate or an antibody oligonucleotide conjugate.

### HIC Materials

A HIC column of the present disclosure may include a HIC material. HIC materials include a non-polar group capable of hydrophobic interactions with one or more analytes passing through the HIC column. The polar group may be, e.g., a monomer within the structure of the HIC material; a shell monomer of a core-shell material; or a surface modification on the surface of the HIC material.

In some embodiments, the HIC material includes an inorganic polymer (e.g., silica), an organic polymer (e.g., a polymer formed from DVB), or a hybrid organic-inorganic polymer. The plurality of HIC particles may include a non-polar surface. The non-polar surface may either be native to the HIC material (e.g., the HIC material is composed of one or more polar groups) or added post synthetically (e.g., via surface modification with a surface moiety including one or more non-polar groups). For example, the surface may be modified to include a hydrophobic group (e.g., an aliphatic hydrocarbon)

The HIC material may include a porous material, a non-porous material, or a superficially porous material. In preferred embodiments, the HIC column includes a porous material. In some embodiments, the porous material is a plurality of porous particles. In some embodiments, the porous material is a porous monolith. In some embodiments, the HIC column includes a superficially porous material. In some embodiments, the superficially porous material is a plurality of superficially porous particles. In some embodiments, the superficially porous material is a superficially porous monolith.

In embodiments wherein the HIC material includes a plurality of superficially porous particles, the HIC material may include a core-shell structure. In some embodiments, the core-shell structure includes a non-porous core and a porous shell. In some embodiments, the core-shell structure includes a superficially porous core and a porous shell. In some embodiments, the superficially porous core includes silica.

In some embodiments, the HIC material includes a plurality of porous particles or a plurality of superficially porous particles. In some embodiments, each particle of the plurality of porous particles or plurality of superficially porous particles includes an average diameter of from 0.5 μm to about 100 μm (e.g., from about 0.5 μm to about 10 μm, e.g., about 1 μm, about 2 μm, about 3 μm, about 4 μm, about 5 μm, about 6 μm, about 7 μm, about 8 μm, about 9 μm, about 10 μm, etc.). In some embodiments, each particle of the plurality of porous particles has an average pore size from about 20 Å to 1500 Å, from about 50 Å to about 1000 Å, from about 60 Å to about 750 Å, or from about 65 Å to about 200 Å.

Exemplary materials which may form an HIC material of the present disclosure include those disclosed in, e.g., U.S. Pat. No. 11,999,808; and U.S. patent application Ser. No. 17/335,539; the materials for hydrophobic interaction chromatography of which are incorporated herein by reference.

### HIC Column Body

The HIC material (e.g., a plurality of HIC particles or an HIC monolith) is typically packed into a chromatographic column, thereby resulting in an HIC column. The column body is typically formed of a metal or a metal alloy, e.g., titanium or stainless steel.

In some embodiments, an alkylsilyl coating or other high-performance surface (HPS) is provided to limit or reduce non-specific binding of a sample with the walls or interior surfaces of the column body. Without wishing to be bound by any particular theory, it is believed than an alkylsilyl coating covering metal surfaces prevents or otherwise minimizes contact between fluids passing through the column. The alkylsilyl coating can be applied to the interior surfaces defining what is known as a wetted path of the column. A metal wetted path includes all surfaces formed from metal that are exposed to fluids during operation of the chromatographic column. The metal wetted path includes not only the column body walls but also metal frits disposed within the column. The coating may be applied not only to the wall of the column body but also to the frits.

In general, the alkylsilyl coating is applied through a vapor deposition technique. Vaporized precursors are charged into a reactor in which the part to be coated is located. These vaporized precursors react on the surfaces of the part to be coated to form a first layer of deposited material. The vapor deposition can be applied in a stepwise function to apply a number of layers of deposited material to the surfaces to grow a thickness of the coating and/or to apply layers of different materials (e.g., alternating between a first and second material) to form the coating.

In some embodiments, the alkylsilyl coating is applied to other portions of the liquid chromatography system. For example, the alkylsilyl coating can be applied to metal components residing upstream and downstream of the column. Specifically, the alkylsilyl coating can be applied to an injector of the liquid chromatography system and to post column tubing and connectors (e.g., tubing and connectors leading from the column to downstream components such as detectors). Further, the affinity chromatographic columns of the present technology do not require the addition of additional organic modifiers to reduce non-specific binding. Typically, the addition of an organic modifier (e.g., acetonitrile) may be necessary with sorbents used in affinity chromatography to reduce non-specific binding. Due to the already low non-specific binding of the columns of the present technology, no organic modifier is necessary.

In one embodiment, the alkylsilyl coating includes a hydrophilic, non-ionic layer of polyethylene glycol silane. In another embodiment, the alkylsilyl coating is formed from one or more of the following precursor materials bis(trichlorosilyl)ethane or bis(trimethoxysilyl)ethane. Other embodiments of alkylsilyl coatings suitable for use with the present technology are described in U.S. Patent Publication No. 2019/0086371 and U.S. Application Publication No. 2022/0118443.

In some embodiments, the HIC column body includes a length of from 50 mm to about 500 mm (e.g., 50 mm, 100 mm, 150 mm, 200 mm, 250 mm, 300 mm, 350 mm, 400 mm, 450 mm, 500 mm, etc.). In some embodiments, the HIC column body includes an internal diameter of from 1 mm to 10 mm (e.g., 2.1 mm, 4.6 mm, etc). In some embodiments, the HIC column body includes a length of 50 mm and an internal diameter of 2.1 mm. In some embodiments, the HIC column body includes a length of 100 mm and an internal diameter of 2.1 mm.

### HIC Buffers

HIC buffers suitable for use in the methods described herein may be characterized by an ionic strength, a representation of the relative charge of the buffer solution. A HIC buffer of a higher ionic strength may be more suitable for separating analytes which interact strongly with a HIC column. A HIC buffer may be further described by, e.g., a pH (e.g., a pH of from 4 to 9, e.g., 4, 5, 6, 7, 8, or 9). In some embodiments, the HIC buffer is compatible with a detection mode. For example, the HIC buffer may be an MS-compatible buffer or a MALS-compatible buffer.

Critically, HIC buffers of the present invention do not cause denaturation or degradation of either components of a sample retained by the affinity chromatography column or the affinity agent of the affinity chromatography column. In some embodiments, any components retained by the affinity chromatography column are not released by the HIC buffer. For example, after a chromatography system of the present disclosure is treated with an affinity buffer, a first HIC buffer may be used which does not cause the release or denaturation of any component still bound to the affinity chromatography column.

Exemplary HIC buffers include those disclosed in e.g., U.S. Pat. No. 11,999,808; and U.S. patent application Ser. No. 17/335,539; the mobile phases and buffers of which are incorporated herein by reference.

### Zero and Low Volume Unions

The present technology and methods utilize an affinity chromatography column and HIC column in direct fluidic connection. The direct fluidic connection can be achieved via a zero dead volume or low volume union. The zero and/or low volume union eliminates the need for the use of a valve switch or sample loop that are typically required to achieve separation of a sample by more than one chromatographic mode of separation.

A zero dead volume union introduces no volume (i.e., about 1 μL or less of volume) to the flow path. A low volume union introduces minimal volume (i.e., between about 0 to about 30 μL of volume) to the flow path.

Zero dead volume unions (also referred to as connectors) and low volume unions (also referred to as connectors) are known in the art. Examples of said unions include the VanGuard™ FIT (available from Waters Technologies Corporation, Milford MA) and as described in U.S. Pat. Nos. 8,449,769 and 9,724,621, incorporated herein by reference.

Additional examples include, but are not limited to, the Sulfinert Zero Dead Volume Union (available from Shimadzu), the Idex High-Pressure Zero Dead Volume Union (available from Fisher Scientific), and the Swagelok® Zero Dead Volume Union (available from Millipore Sigma).

Zero dead volume and low volume unions include male-male unions, female-female unions, and male-female unions, and can be used to connect the affinity chromatography column directly to the HIC column.

### Chromatography Systems

The chromatography systems described herein include an affinity chromatography column in direct fluidic connection to a HIC column. The direct fluidic connection between the affinity chromatography column and the HIC column is via a zero dead volume or low volume union. The chromatography system may further include a solvent pump, solvent mixer, a sample injector, and one or more detectors.

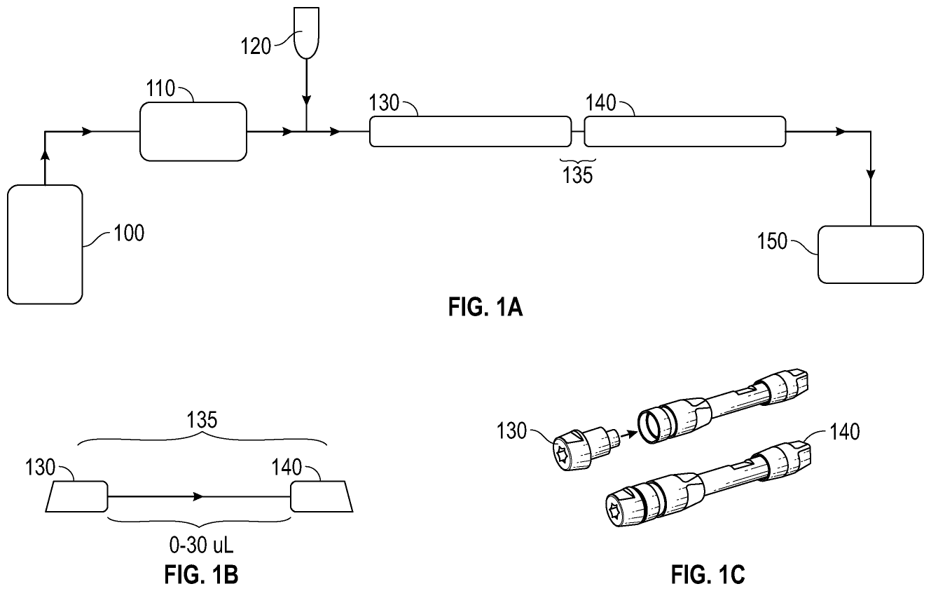

FIGS. 1A-1C depict an exemplary liquid chromatography (LC) system of the present technology. FIG. 1A demonstrates the LC system, with the flow path represented by the arrows. Solvent pump (100) pumps the solvent to the solvent mixer (110). A sample, which includes the target analyte, is loaded into the system using a sample injector (120) and flowed through the affinity chromatography column (130). The target analyte is eluted from the affinity chromatography column (130) directly onto the HIC column (140). Affinity chromatography column (130) and HIC column (140) are in direct fluidic connection via a zero dead volume or low volume union (135). There is no intervening component between the affinity chromatography column (130) and the HIC column (140) except for the union (135) and possibly tubing. That is, no valves or other components exist between column (130) and column (140). Once eluted from the HIC column, the sample can be analyzed via a detector (150). For example, while FIG. 1A depicts a single detector (150), it is understood that the single detector (150) may be absent, or there may be multiple detectors connected in fluidic series.

FIG. 1B provides an enlarged view of the direct fluidic connection between the affinity chromatography column (130) and the HIC column (140). The flow path of 135 is between 0-30 μL (microliters) of volume. In some embodiments, the flow path of 135 is less than 1 μL.

FIG. 1C depicts an exemplary union between the affinity chromatography column (130) and the HIC column (140).

The chromatography systems described herein allow for direct affinity-HIC in the absence of valve switching components. Accordingly, the present disclosure features chromatography systems without a valve switch along the flow path of the chromatography system. The chromatography systems described herein further allow for direct affinity-HIC in the absence of trap configurations. Accordingly, in some embodiments, a trap is absent from the flow path of the chromatography system.

In some embodiments, the size HIC column (140) is connected in fluidic series to one or more detectors. Detectors suitable for use in the methods disclosed herein include detectors for ultraviolet spectroscopy, fluorescence spectroscopy, mass spectrometry, and/or other detectors used for the characterization of analytes, such as, for example, multi-angle light scattering (MALS) detectors and charged aerosol detectors (CAD). In some embodiments, the liquid chromatography system is connected in series to a detector for ultraviolet spectroscopy. In some embodiments, the liquid chromatography system is connected in series to a detector for fluorescence spectroscopy. In some embodiments, the liquid chromatography system is connected in series to detector for mass spectrometry. In some embodiments, the liquid chromatography system is connected in series to a MALS detector. In some embodiments, the liquid chromatography system is connected in series to a CAD. In some embodiments, the liquid chromatography system is connected to one or more of the detectors in series.

### Methods of Use

The methods disclosed herein pertain to direct affinity-HIC for the purification and characterization of a target analyte from a sample. The method includes loading a sample including the target analyte onto an affinity chromatography column, washing the affinity chromatography column with an affinity buffer, eluting the target analyte from the affinity chromatography column directly onto the HIC column, and eluting the target analyte from the HIC column. In embodiments wherein the HIC column is connected in fluidic series to one or more detectors, the methods further include detecting the target analyte using the one or more detectors (e.g., a UV detector, a fluorescence detector, a MALS detector, and/or an MS detector). As such, the direct affinity-HIC methods described herein afford the analysis of a sample across two modes of separation, including the analysis of peaks that do not bind to the affinity column (i.e., flow through the column in the presence of the first affinity buffer, i.e. “load peaks”) and peaks that elute from the affinity column in the presence of the second affinity buffer (i.e., “elution peaks”).

Methods of the present disclosure may employ one or more buffers (e.g., affinity buffers, HIC buffers) during a step of a method. An affinity buffer may be employed during an affinity chromatography step to control the binding of one or more analytes to the affinity chromatography column. For example, a first affinity buffer may be employed to separate a collection of analytes with affinity for the chromatography column from a collection of analytes without affinity for the affinity chromatography column. A second affinity buffer may later be used to reduce the affinity of the analytes to the affinity chromatography column, thereby allowing the analytes to be eluted from the affinity chromatography column to the HIC column. A first HIC buffer may be employed during an HIC step to control the binding of one or more analytes to the HIC column. A second HIC buffer may be later used to modify the interaction between one or more analytes and the HIC column, thereby improving the efficiency of the separation step. In some embodiments, the first affinity buffer may also serve as the first HIC buffer, In some embodiments, the second affinity buffer may also serve as the second HIC buffer. For example, the affinity chromatography buffer used in the injection of a sample onto the affinity chromatography column may also be suitable for separating one or more components of the flowthrough from the affinity chromatography column on the HIC column. Therefore, the same buffer can be used in both steps.

Each buffer may contain one or more specific components to aid in the performance of a method step. For example, an affinity bugger may include an acid (e.g., phosphoric acid) at a concentration sufficient to modify the affinity of an analyte to an affinity chromatography column. As a further example, an HIC buffer may include a salt (e.g. sodium chloride) at a concentration sufficient to modify the affinity of an analyte to an affinity chromatography column.

In some embodiments, the skilled artisan may select second affinity buffer or a second HIC buffer such that one or more analytes remain associated with the affinity chromatography column or the HIC column after the elution step (e.g., an analyte with sufficient affinity for the affinity chromatography column that it was not released by the first affinity buffer or the second affinity buffer; and/or an analyte with sufficient affinity for the HIC column that it was not released by the first HIC buffer or the second HIC buffer). These retained analytes may be eluted from the chromatography column by repeating the steps of injecting an additional affinity buffer and/or injecting an additional HIC buffer an appropriate number of times. In some embodiments, each subsequent affinity buffer may be configured to reduce the binding affinity of the analyte to the affinity chromatography column. For example, each affinity buffer may include an increased concentration of acid (e.g., an increased concentration of phosphoric acid); or each HIC buffer may include an increased ionic strength (e.g., an increased concentration of sodium chloride). In preferred embodiments, methods of the present disclosure include no more than two affinity buffers. In some embodiments, the second affinity buffer and the second HIC buffer may be the same or similar buffer compositions. This step may be repeated as many times as necessary to release the desired analyte from the chromatography system.

In some embodiments, one or more HIC elution steps may be performed for each affinity elution step. For example, an HIC elution step may be sufficient to elute a first analyte from the HIC chromatography column, but insufficient to remove one or more additional analytes from the chromatography column. In some embodiments, each HIC elution step may be performed with a unique HIC buffer (e.g., a first HIC buffer, a second HIC buffer, a third HIC buffer, etc.). Each successive HIC buffer may be characterized by an increasing ionic strength (e.g., an increasing concentration of sodium chloride), thereby improving the ability of each successive HIC elution step to remove an analyte from the HIC chromatography column. Each affinity elution step may include one or more HIC elution steps.

In some embodiments, the HIC buffer or HIC buffers used in an affinity elution step may be identical. For example, the first HIC buffer injected into the chromatography system after the first affinity elution step, and the first HIC buffer injected into the chromatography system after the second affinity elution step may be identical. In some embodiments, the HIC buffer or HIC buffers used in an affinity elution step may be unique.

One or more target analytes can be eluted from the affinity chromatography column or the HIC column using various elution methods. Exemplary methods include gradient elution, step elution, or injection elution. Gradient elution methods involve the transition of the mobile phase from a first solvent condition (e.g., the mobile phase, the first affinity buffer, or the first HIC buffer) to a second solvent condition elution buffer over a period of time (e.g., 1-3 minutes), thereby forming a gradient of the elution buffer that flows through the column (ranging from 0-100% of the second solvent condition). In some embodiments, the method may include applying a gradient elution of the affinity buffer to elute one or more analytes from the affinity chromatography column. In some embodiments, the method may include applying a gradient elution of the HIC buffer to elute one or more analytes from the HIC column.

An injection elution method involves a single injection of a buffer (e.g., the affinity buffer or the HIC buffer) into the mobile phase. As used herein, “single injection a buffer” (also described as a “single injection elution” or an “injection elution”) refers to a one-time injection of a specific volume of a buffer, for example, a single injection of 1 μL (or 10 μL, 20 μL, 30 μL, 40 μL, 50 μL, or any volume between 1-50 μL) of the buffer into the affinity chromatography column. In other embodiments, the single injection of the buffer has a volume that is between 50-100% of the column volume. For example, with a column having a column volume of 1 mL, the single injection of may be between 500 μL to 1 mL, or any volume in between (e.g., 600 μL, 700 μL, 800 μL, 900 μL). The single injection of a buffer can be achieved by injecting the buffer into the flow path of the affinity chromatography column using a sample injector. Alternatively, a switch valve upstream of the affinity chromatography column can be used to perform the single injection of the buffer. The single injection is performed such that the volume of elution buffer bypasses or otherwise does not flow through a mobile phase mixer of a liquid chromatography system. In some embodiments, the method may include applying an injection elution of the affinity buffer to elute one or more analytes from the affinity chromatography column. In some embodiments, the method may include applying an injection elution of the HIC buffer to elute one or more analytes from the HIC column.

An exemplary method of the present disclosure is now described. First, a sample is loaded onto the affinity chromatography column in the presence of a first affinity buffer. The affinity chromatography column is then washed with the first affinity buffer. Analytes with an affinity for the affinity chromatography column are retained by the affinity chromatography column, and analytes without affinity for the affinity chromatography column are eluted to the HIC column as the load peak described above. In some embodiments, the flowthrough from the affinity chromatography column is further separated by the HIC column. In some embodiments, a first HIC buffer may be injected into the chromatography system to modify the interaction of one or more analytes in the flowthrough and the HIC material, allowing the separation of the one or more analytes by HIC. In such embodiments, the first HIC buffer does not degrade or denature any analytes retained by the HIC chromatography column. In some embodiments, the first HIC buffer does not degrade or denature the affinity agent on the surface of the affinity chromatography material.

A second affinity buffer is then injected into the chromatography system. The second affinity buffer is configured to release one or more analytes bound to the affinity chromatography column in the presence of the first affinity buffer from the column, allowing the one or more analytes to flow through the affinity chromatography column onto the HIC chromatography column as the elution peak described above. In some embodiments, the eluent from the affinity chromatography column is further separated by the HIC column in the presence of the second affinity buffer. In some embodiments, a second HIC buffer may be injected into the chromatography system to modify the interaction of one or more analytes in the flowthrough and the HIC material, allowing the separation of the one or more analytes by HIC.

As the flow path between the affinity chromatography column and the HIC column is uninterrupted, the buffers used in the provided methods are compatible with both columns. Thus, the methods provide for direct affinity-HIC methods without solvent mismatch or the need for buffer exchange between modes of separation. Further, as the buffers used for both columns are the same, the disclosed methods eliminate or otherwise minimize the need for column equilibration between samples.

### EXAMPLES

### Example 1: Exemplary Method of Using a Direct Affinity-HIC Chromatography System

The following example describes the characterization of an exemplary sample (e.g., an immunoglobulin sample, an antibody sample, an oligonucleotide sample, etc.) using a direct affinity-hydrophobic interaction (direct affinity-HIC) chromatography system. The affinity chromatography column is composed of a plurality of nonporous polymer particles with an average size of from about 1 μm to about 10 μm. The plurality of nonporous polymer particles within the affinity chromatography column has a hydrophilic layer on its outer surface. The plurality of nonporous polymer particles includes an affinity agent (e.g., an immunoglobulin-binding affinity agent, an antibody affinity agent, or an oligonucleotide affinity agent). The hydrophobic interaction chromatography column is composed of a plurality of HIC particles with an average particle size of from about 1 μm to about 10 μm. The plurality of HIC particles includes a hydrophobic surface. The affinity chromatography column and the HIC column are joined by either a zero dead volume union or a low dead volume union.

The sample is loaded onto the affinity chromatography column, and the affinity chromatography column is washed with a first affinity buffer. Components of the sample with an affinity for the affinity chromatography column are retained by the affinity chromatography column, and analytes without affinity for the affinity chromatography column are eluted through the affinity column onto the HIC column. A first HIC buffer is then injected into the chromatography system. The first HIC buffer is configured to not interact with the components of the sample which remain bound to the affinity chromatography column (e.g., protein components of the sample are not denatured by the first HIC buffer, all components retained by the affinity chromatography column remain retained by the affinity chromatography column, etc.). After passing through the affinity chromatography column, the first HIC buffer interacts with the components of the sample which eluted from the affinity chromatography column onto the HIC column. These components of the sample are then separated by the HIC chromatography column. In order to better separate these components using HIC chromatography, a gradient mobile phase may be used.

After elution of the components in the HIC chromatography column is complete, a second affinity buffer is then injected into the chromatography system. The second affinity buffer is configured to release one or more of the components previously retained by the affinity chromatography column, allowing the one or more previously retained components to flow through the affinity chromatography column onto the HIC chromatography column. A second HIC buffer is then injected into the chromatography system. The second HIC buffer then interacts with the previously retained components in the HIC chromatography column, thereby allowing separation of the previously retained components by HIC chromatography. As with the first HIC buffer, a gradient mobile phase may be used to better separate the previously retained components.

It is contemplated that in some embodiments, the second affinity buffer and the second HIC buffer may be the same buffer. In other words, a buffer used to dissociate any retained analytes from an affinity chromatography column may further serve as the HIC buffer in the HIC step of the method.

1. A method of purifying a target analyte, the method comprising:
a) loading a sample comprising the target analyte onto an affinity chromatography column in direct fluidic connection to a hydrophobic interaction chromatography (HIC) column, the affinity chromatography column comprising:
a plurality of nonporous polymer particles, wherein each particle within the plurality of nonporous polymer particles comprises a polymer core and a hydrophilic surface on an outer layer of the polymer core; and
one or more affinity agents conjugated to the hydrophilic surface of each particle of the plurality of nonporous polymer particles;
and the HIC column comprising:
a plurality of HIC particles having a non-polar surface;
b) eluting the target analyte from the affinity chromatography column with an affinity buffer directly onto the HIC column; and
c) eluting the target analyte from the HIC column with an HIC buffer.

2. The method of claim 1, wherein the one or more affinity agents are conjugated directly to a surface of each particle of the plurality of nonporous polymer particles.

3. The method of claim 2, wherein the one or more affinity agents are conjugated to the surface of each particle of the plurality of nonporous polymer particles covalently.

4. The method of claim 1, wherein the one or more affinity agents are conjugated indirectly to the surface of each particle of the plurality of nonporous polymer particles through one or more conjugating molecules.

5. The method of claim 4, wherein the one or more conjugating molecules comprise streptavidin.

6. The method of claim 5, wherein the one or more affinity agents are biotinylated.

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. A chromatographic system comprising:
(a) an affinity chromatography column and a hydrophobic interaction chromatography (HIC) column, wherein the affinity chromatography column and the HIC column are connected via a zero dead volume union, wherein the HIC column is positioned downstream of the affinity chromatography column; wherein the affinity chromatography column comprises:
a plurality of nonporous polymer particles, wherein each particle within the plurality of nonporous polymer particles comprises a polymer core and a hydrophilic surface on an outer layer of the polymer core; and
one or more affinity agents conjugated to the hydrophilic surface of each particle of the plurality of nonporous polymer particles;
and wherein the HIC column comprises:
a plurality of HIC particles having a non-polar surface;
(b) a column injector positioned upstream of the affinity chromatography column; and
(c) tubing in fluidic connection with the HIC column positioned downstream of the HIC column.

27. The chromatographic system of claim 26, wherein the one or more affinity agents are conjugated directly to a surface of each particle.

28. The chromatographic system of claim 27, wherein the one or more affinity agents are conjugated to the surface of each particle covalently.

29. The chromatographic system of claim 26, wherein the one or more affinity agents are conjugated indirectly to the surface of each particle through one or more conjugating molecules.

30. The chromatographic system of claim 29, wherein the one or more conjugating molecules comprise streptavidin.

31. The chromatographic system of claim 30, wherein the one or more affinity agents are biotinylated.

32. The chromatographic system of claim 26, wherein the one or more affinity agents comprise an immunoglobulin-binding protein, an antibody or antigen-binding fragment thereof, or an oligonucleotide.

33. The chromatographic system of claim 32, wherein the immunoglobulin-binding protein is Protein A, Protein G, Protein A/G, Protein L, or a binding domain thereof.

34. The chromatographic system of claim 32, wherein the antibody or antigen-binding fragment thereof binds to insulin, an AAV capsid, tacrolimus, troponin, IgG, a cytokine, a host cell protein, a dsRNA, or perfluoroalkyl substances (PFAS).

35. The chromatographic system of claim 34, wherein the AAV capsid is AAV1, AAV2, AAV3, AAV4, AAV5, AAV6, AAV7, AAV8, AAV9, AAVrh10, or a synthetic serotype thereof.

36. The chromatographic system of claim 32, wherein the oligonucleotide is a poly-T oligonucleotide.

37. The chromatographic system of claim 26, wherein the plurality of nonporous polymer particles have an average particle size between 1.0 μm to 10 μm.

38. The chromatographic system of claim 26 wherein the plurality of HIC particles have an average particle size between 1.0 μm to 10 μm.

39. The chromatographic system of claim 26, further comprising a detector in fluid communication with the tubing downstream of the HIC chromatography column.
